(19) United States
(12) Patent Application Publication
(10) Pub. No.: US 2007/0292384 A1
(43) Pub. Date: Dec. 20, 2007

(54) Water soluble micelle-forming and biodegradable cyclotriphosphazene-taxol conjugate anticancer agent and preparation method thereof

(76) Inventors: Youn-Soo Sohn, Seoul (KR); Da-Eun Ji, Seoul (KR); Yong-joo Jun, Seoul (KR); Hwa-Jeong Lee, Seoul (KR)

Correspondence Address: MORRISON & FOERSTER LLP, 12531 HIGH BLUFF DRIVE, SUITE 100, SAN DIEGO, CA, 92130-2040, US

(21) Appl. No.: 11/818,744

(22) Filed: Jun. 15, 2007

(30) Foreign Application Priority Data
Jun. 16, 2006 (KR) 10-2006-0054548

(51) Int. Cl. A61K 31/78; C08G 79/02

(52) U.S. Cl. 424/78.27; 525/538; 977/906

(57) ABSTRACT

A water-soluble micelle-forming cyclotriphosphazene-taxol conjugate anticancer agent, which is biodegradable, and thus, slowly release taxol, represented by the following Formula 1:

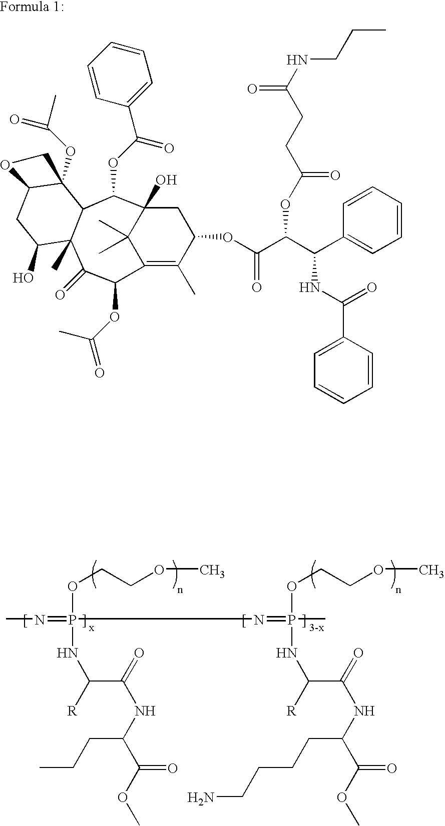

wherein R is selected from the group consisting of H, CH₃, (CH₃)₂CH and (CH₃)₂CHCH₂; each n is the number of ethylene oxide repeating unit of poly(ethylene glycol) selected from the integers of 7-12; and x is 1 or 2.

### CROSS-REFERENCE TO RELATED APPLICATIONS

This application claims priority to Korean Application No. 10-2006-0054548 filed 16 Jun. 2006. The contents of the above patent application is incorporated by reference herein in its entirety.

### TECHNICAL FIELD

The present invention relates to a cyclotriphosphazene-taxol conjugate anticancer agent which forms water soluble micelles and which can slowly degrade and release taxol in vivo, and to a preparation method thereof.

### BACKGROUND OF THE INVENTION

A phosphazene-based polymer, polyphosphazene, is an inorganic/organic hybrid polymer which was first synthesized by Allcock Group in the United States (H. R. Allcock, R. L. Kugel, J. Am. Chem. Soc. (1965) 87:4216). It is a linear polymer in which its backbone consists of alternating phosphorus and nitrogen atoms, and organic substituents are grafted to the phosphorus atoms as side groups. Polyphosphazenes exhibit a variety of different physicochemical properties depending on the molecular structure of their side chains. Even though polyphosphazenes have good physical properties that organic polymers do not have, they could not have been used as general-purpose polymer materials but only used for limited purpose. In particular, polyphosphazenes have not been developed as drug delivery systems because the polymeric drug delivery materials require a low molecular weight (Mw=10⁴⁻⁵) for biocompatibility as well as biodegradability, while the conventional polyphosphazenes were developed for general-purpose polymers requiring a higher molecular weight (Mw>10⁶) for high mechanical strength.

The present inventors discovered that the molecular weight of polyphosphazenes could be controlled by the amount of aluminum chloride used as a catalyst for thermal polymerization reaction of hexachlorocyclotriphosphazene (N₃P₃Cl₆) to produce poly(dichloro-phosphazene) (Youn Soo Sohn, et al., Macromolecules (1995) 28:7566). Based on this discovery, the present inventors have been involved in the development of various new polymeric drug delivery systems. However, in recent years, the present inventors have successfully synthesized a new class of thermosensitive cyclotriphosphazenes instead of polyphosphazenes for the first time by direct nucleophilic substitutions of hexachlorocyclotriphosphazene (N₃P₃Cl₆) with equimolar amounts of a hydrophilic poly(ethylene glycol) and a hydrophobic amino acid at a low temperature (e.g., at −60° C.) (Youn Soo Sohn, et al., J. Am. Chem. Soc. (2000) 122:8315). However, we found that these cyclic trimers bearing amino acids as a hydrophobic group did not form micelles probably because of the low hydrophobicity of amino acids and are unstable in aqueous solution, which rendered their practical applications difficult.

Most recently, the present inventors have discovered that if a tri- or tetra-oligopeptide having much higher hydrophobicity compared with amino acids is introduced into the trimer along with a poly(ethylene glycol), stable micelles with a mean diameter of 10-100 nm are formed by self-assembly in aqueous solution, and thus, obtained a patent as Korean Patent No. 567397. The present inventors have been currently conducting researches to apply such thermosensitive micelles to a formulation of hardly soluble drugs such as protein drugs, taxol, or the like.

According to the recent clinical studies, taxol exhibits outstanding effects for the treatment of various types of cancers such as breast cancer, ovarian cancer, small cell lung cancer and the like (E. K. Rowinsky, R. C. Donehower, New Engl. J. Med. (1995) 332:1004-1014). Accordingly, taxol is the most widely used anticancer agent. However, since taxol is highly hydrophobic and hardly soluble in water (<1 μg/ml), it cannot be directly injected as an aqueous solution. Therefore, taxol is formulated with a surfactant (Cremophore EL) and ethanol as solubilizing agents, which cause severe adverse effects such as neurotoxicity (R. T. Dorr, Ann. Pharmacother. (1994) 28:S11-S14).

Accordingly, various studies have been conducted to solve the aforementioned problems. In particular, researches for using polymer micelles have actively been conducted (K. M. Huh, et al. J. Control. Release (2005) 101:59-68; O. Soga, et al. J. Control. Release (2005) 103:341-353). In polymer micelles composed of both hydrophilic and hydrophobic blocks, the inner core of the micelles consists of hydrophobic groups, which can efficiently trap hydrophobic drugs such as taxol inside the micelles, by which the hydrophobic drugs can be solubilized. There are also many other research reports on conjugate drugs such as a conjugate anticancer agent which is solubilized by conjugating taxol molecule directly with a hydrophilic poly(ethylene glycol) (R. B. Greenwald, et al., Advanced Drug Delivery (2003) 55:217-250 and M. Ceruti, et al., J. Control. Release (2000) 63:141-153) and a polymeric taxol anticancer agent, in which taxol is conjugated to a water-soluble polyglutamic acid (C. Li, et al., Cancer Chemother. Pharmacl. (2000) 46:416-422). Especially, those polymeric anticancer agents are currently in clinical trials. However, no example has been reported for taxol which is conjugated to or formulated with a phosphazene-based polymer. Furthermore, there is no report on taxol anticancer agent conjugated to a hydrophilic polymer that forms micelles by self-assembly in an aqueous solution.

### SUMMARY OF THE INVENTION

Therefore, an object of the present invention is to provide a novel water-soluble cyclotriphosphazene-taxol conjugate anticancer agent capable of reducing the adverse effects caused by surfactant Cremophore/alcohol used as solubilizing agents in formulating taxol, which is a practically insoluble anticancer agent clinically used, thereby making it possible to fundamentally overcome the problems of conventional treatment procedures which make a patient hospitalized for infusion for a long time.

Another object of the present invention is to provide a method for preparing the cyclotriphosphazene-taxol conjugate anticancer agent.

### BRIEF DESCRIPTION OF THE DRAWINGS

The accompanying drawings, which are included to provide a further understanding of the invention and are incorporated in and constitute a unit of this specification, illustrate embodiments of the invention, and together with the description serve to explain the principles of the invention.

In the drawings:

FIG. 1 shows the particle size distribution, which was measured by dynamic light scattering (DLS) method, of an aqueous solution in which the cyclotriphosphazene-taxol conjugate prepared in Example 1 of the present invention is dissolved in distilled water at a concentration of 0.1 to 0.5%.

FIG. 2 shows experimental results of the hydrolysis rate for the cyclotriphosphazene-taxol conjugate prepared in Example 1 of the present invention.

### MODES OF CARRYING OUT THE PREFERRED EMBODIMENTS

The present invention provides a water-soluble conjugate anticancer agent which is prepared by introducing as a hydrophilic substituent a poly(ethylene glycol) (PEG) having a molecular weight of 350 or more into the cyclotriphosphazene trimer, followed by introducing as a spacer group an oligopeptide having a relatively lower hydrophobicity, especially a lysine-containing dipeptide, for example, glycyllysine methyl ester, and then chemically coupling succinyl taxol with an ε-amine group of lysine of the space group. The present inventors have discovered that the conjugate anticancer agent according to the present invention is immediately soluble in water and is capable of forming water-soluble micelles having a mean diameter of 10-150 nm, and thus, when it is administered intravenously, it has longer blood circulation time compared with free taxol, and taxol is slowly released from the cores of the micelles by slow biodegradation, resulting in sustained and continuous pharmacological effects, by which the adverse effects due to drugs can be drastically reduced and bioavailability of the taxol can be maximized.

Therefore, the first aspect of the present invention relates to a water-soluble micelle-forming cyclotriphophazene-taxol conjugate anticancer agent, capable of slowly releasing taxol while it is degraded in vivo, represented by the following Formula 1:

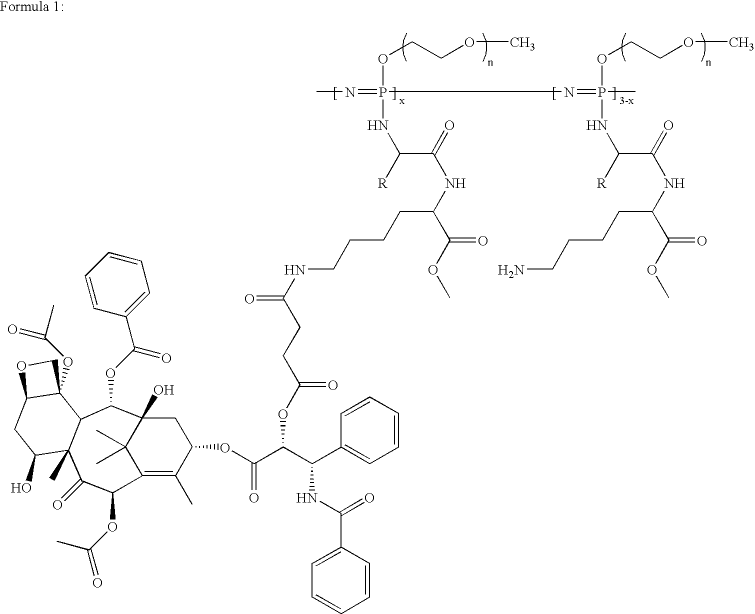

wherein R is selected from the group consisting of H, CH₃, (CH₃)₂CH and (CH₃)₂CHCH₂; each n is the number of ethylene oxide repeating unit selected from the integers of 7-12; and x is 1 or 2.

The second aspect of the present invention relates to a method for preparing the water-soluble micelle-forming cyclotriphophazene-taxol conjugate anticancer agent represented by Formula 1 above. The cyclotriphophazene-taxol conjugate anticancer agent according to the present invention can be prepared by the method described below. According to the present invention, it is preferable to use thoroughly-dried reagents and solvents and to perform all the preparation procedures under inert atmosphere so as to avoid even a trace of moisture.

The preparation method of the compound of Formula 1 according to the present invention comprises:

(1) reacting a poly(ethylene glycol) monomethyl ester (MPEG) represented by Formula 2 with sodium or sodium hydride to obtain a sodium salt of poly(ethylene glycol) represented by Formula 3, followed by reacting the obtained sodium salt with hexachlorocyclotriphosphazene (N₃P₃Cl₆) of Formula 4 to obtain a cyclotriphosphazene intermediate represented by Formula 5:

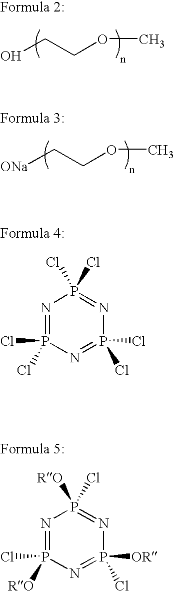

(2) reacting the obtained cyclotriphosphazene intermediate of Formula 5 with methyl ester of glycyllysine represented by Formula 6 to obtain a compound represented by Formula 7, followed by deprotecting the compound of Formula 7 to obtain a compound represented by Formula 8; and

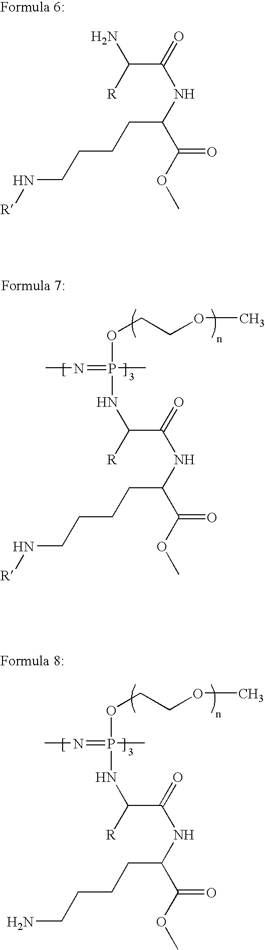

(3) reacting the compound of Formula 8 with succinyl taxol to obtain the cyclotriphosphazene-taxol conjugate of Formula 1.

R″ in Formula 5 is CH₃(OCH₂CH₂)ₙ; R′ in Formula 6 is benzyloxycarbonyl group or t-butoxycarbonyl group; and n in Formulas 2 to 8 is the same as that defined in Formula 1.

Hereinafter, the method for preparing the cyclotriphosphazene-taxol conjugate of Formula 1 is described in more detail.

In Step (1), it is preferred that poly(ethylene glycol) monomethylester (MPEG) is dried under a vacuum condition in an oil bath at 70-80° C. for 1 or 2 days prior to use, so as to remove moistures therefrom. In preparing the sodium salt of a compound of Formula 2, it is appropriate to use 1.1-1.5 equivalents of sodium or sodium hydride with respect to 1 equivalent of MPEG of Formula 2. Tetrahydrofuran (THF), benzene or toluene may be used as a reaction solvent, in which 3-4 equivalents of sodium salt of Formula 3 are dissolved and then slowly added to the solution of 1 mol of hexachlorocyclotriphosphazene (6 equiv.) of Formula 4 in the same solvent containing excess triethylamine in a reaction vessel kept at dry ice-acetone temperature (−60° C.). The reaction mixture is further stirred for 1-2 hours and then warmed up to room temperature. In Step (1), the cyclotriphosphazene intermediate of Formula 5 is obtained as an isomer with cis-nongeminal conformation, in which three poly(ethylene glycol) groups are oriented in the same side with respect to the phosphazene ring.

In Step (2), the cyclotriphosphazene intermediate of Formula 5 obtained in Step (1) is reacted with 1.1-1.5 equivalents of glycyllysine of Formula 6 for each of three un-substituted chlorine atoms in the cyclotriphosphazene intermediate of Formula 5 by adding a solution in which glycyllysine of Formula 6 and 4 equivalents of triethylamine are dissolved in chloroform or methylene chloride. The resulting mixture is reacted at room temperature for one day and then refluxed at a temperature of 40-60° C. for 1-2 days. The reaction solution is then purified by the following method so as to isolate a pure compound of Formula 7. First, the reaction solution is centrifuged or filtered to remove precipitated byproducts (Et₃N.HCl or NaCl). The filtrate is concentrated under a reduced pressure, and the residue is dissolved in tetrahydrofuran, and an excessive amount of ethyl ether or hexane is then added so as to induce precipitation. This process is repeated twice. The precipitate is dissolved in a small amount of distilled water and subjected to dialysis using a dialysis membrane (MWCO: 1000) for 1 or 2 days, and then the dialyzed solution is freeze-dried. Next, the resultant product is dissolved in distilled water. The aqueous solution is warmed up to its lower critical solution temperature (LCST) at which the pure trimeric product precipitates. The resultant precipitate is recovered by centrifugation. After this process is also repeated twice, the aqueous solution is freeze-dried to give a pure cyclotriphosphazene of Formula 7, which is further reacted with a methanol solution containing 5-10 equivalents of cyclohexadiene for each dipeptide group of cyclotriphosphazene of Formula 7 using palladium charcoal as catalyst to remove the benzyloxycarbonyl or t-butoxycarbonyl group, resulting in the compound of Formula 8.

In Step (3), the compound of Formula 8 is dissolved in methylene chloride or tetrahydrofuran, and 3 equivalents of triethylamine are added thereto. The resultant solution is cooled down to −78° C. using a dry ice-acetone bath and stirred for the reaction with succinyl taxol. In a separate container, 1 or 2 equivalents of 2′-succinyl taxol for 1 mole of the compound of Formula 8 are dissolved in methylene chloride or tetrahydrofuran. An amide coupling reagent, for example, a solution of the same equivalents both of 1-ethyl-3-(3-dimethylaminopropyl) carbodiimide (EDC) and 1-hydroxybenzonitroazole hydrate (HOBt) as those of 2′-succinyl taxol dissolved in methylene chloride is slowly added dropwise to the above 2′-succinyl taxol solution, and the resultant solution is then refluxed. In addition to the aforementioned coupling reagent, there are various amide coupling reagents which are commonly used in the art. Thus, the aforementioned amide coupling reagent is a merely exemplary, and it is noted that the same purpose of the present invention can be achieved using any other reagents known in the art. The 2′-succinyl taxol reaction solution thus obtained is slowly added to the previously prepared cyclotriphosphazene solution of Formula 8, and the resulting reaction mixture is refluxed until the reaction is completed. The final reaction solution is filtered to remove precipitates, and the filtrate is evaporated under a reduced pressure and freeze-dried, thereby finally obtaining cyclotriphosphazene-taxol conjugate of Formula 1.

The following Reaction Scheme 1 illustrates exemplary overall synthetic procedures for preparing the compound of Formula 1 according to the present invention.

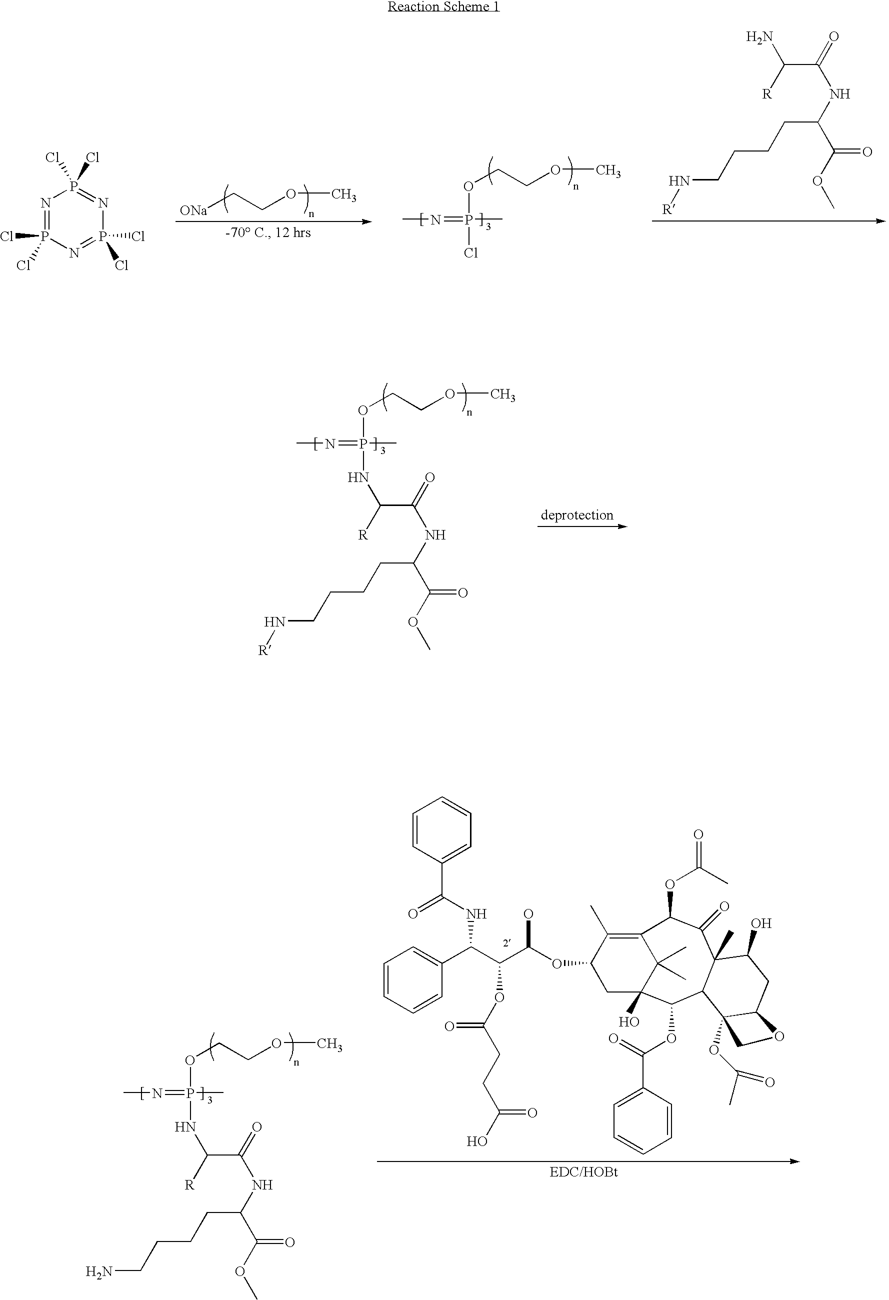

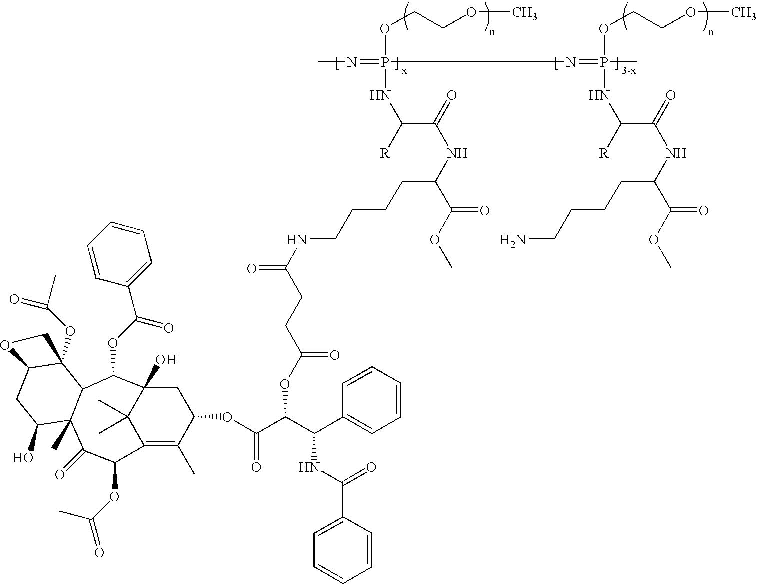

Reaction conditions such as a reagent, a solvent, a reaction time or temperature, and the like, which are indicated in Reaction Scheme 1 are merely exemplary for preparing the compound of Formula 1 according to the present invention. Therefore, it is noted that in preparing the compound of Formula 1 according to Steps (1) to (3) previously described, the reaction conditions can be modified from those indicated in Reaction Scheme 1, and such modifications are not departed from the scope of the present invention.

### EXAMPLES

Hereinafter, the present invention will be described in more detail with reference to examples. However, examples are intended to be merely illustrative, and the scope of the present invention may not be limited thereto without departing from the appended claims.

The elementary analysis of carbon, hydrogen and nitrogen for the compounds of the present invention was performed with Perkin-Elmer C, H, N analyzer. Hydrogen nuclear magnetic resonance spectra were obtained with Bruker DPX-250 NMR spectrometer, and phosphorous nuclear magnetic resonance spectra were obtained with Varian Gemini-400 NMR spectrometer.

### Example 1

### Preparation of {[tris(methoxy-polyethyleneglycol350)][tris(glycyllysinemethylester)-mono-2′-succinyltaxol]cyclotriphosphazene}, [N₃P₃(MPEG350)₃(GlyLys-Me-2′-succinyltaxol)(GlyLysMe)₂]

Under an argon atmosphere, methoxy-poly(ethylene glycol) having a molecular weight of 350 (MPEG350) (3.32 g, 9.50 mmol) and sodium hydride (0.24 g, 9.98 mmol) were dissolved in dry tetrahydrofuran, and stirred for 4 hours to obtain the sodium salt of MPEG350. This sodium salt solution in tertahydrofuran was slowly added for 30 minutes to a solution of hexachlorocyclotriphosphazene (N₃P₃Cl₆, 1.00 g, 2.28 mmol) dissolved in the same solvent under a dry ice-acetone bath (−78° C.). After 30 minutes, the dry ice-acetone bath was removed, and the reaction was continued at room temperature for 8 hours.

Glycyl(benzyloxycarbonylysine) methyl ester (Gly(cbz)LysMe) (3.57 g, 11.52 mmol), which was neutralized with triethylamine (3.58 ml, 25.80 mmol) in dry chloroform (100 mL) in a separate reaction vessel, was slowly added to the previously prepared PEGylated cyclotriphosphazene solution, and the reaction was continued at room temperature for 24 hours. The reaction solution was filtered to remove the precipitated byproducts (i.e., NEt₃.HCl or NaCl), and the filtrate was concentrated under a reduced pressure. The residue dissolved in a small amount of water was dialyzed using a dialysis membrane (MWCO: 1000) for 24 hours, and then freeze-dried. The resultant product was dissolved in tetrahydrofuran, and then recrystallized by adding an excess amount of ethyl ether or hexane thereto. The solid product was then dissolved in methanol (100 ml), and palladium charcoal (3 g) and cyclohexadiene (1.46 ml, 14.1 mmol) were added to the resulting solution, which was further reacted under a nitrogen atmosphere for 48 hours, thereby removing benzyloxycarbonyl group as an amine protective group of lysine to give a cyclotriphosphazene [NP(MPEG350)GlyLysMe]. In order to remove a small amount of other isomers, this product was dissolved in 20 ml of distilled water, which was slowly warmed up for cloud point separation by precipitation at its LCST of 38° C. This recrystallization process was repeated twice, and the resulting solution was freeze-dried to obtain a pure cyclotriphosphazene drug carrier, [NP(MPEG350)GlyLysMe]₃ (65% of yield).

Thus obtained cyclotriphosphazene drug carrier (500 mg, 0.29 mmol) was dissolved along with triethylamine (NEt₃) (0.09 ml, 0.67 mmol) and 2′-succinyl taxol (270 mg, 0.29 mmol) in methylene chloride in a reaction vessel. To this reaction solution kept in a dry ice-acetone bath (−78° C.) was slowly added a solution of the amide coupling agents, EDAC and HOBt dissolved in methylene chloride, and the resultant mixture was stirred for an hour. After the dry ice-acetone bath was removed, the solution was further stirred at room temperature for 48 hours. The reaction solution was evaporated under a reduced pressure, and then the residue was dissolved in a small amount of methanol, which was dialyzed in water using a dialysis membrane (MWCO: 1000) and then freeze-dried to obtain the final product, [N₃P₃(MPEG350)₃(GlyLys-Me-2′-succinyltaxol)(GlyLysMe)₂] (62% of yield).

Empirical formula: C₁₂₃H₁₉₈N₁₃O₅₀P₃

Molecular weight: 2,784

Elementary analysis (%)

Found: C, 54.63; H, 7.17; N, 6.04

Calculated (%): C, 54.35; H, 7.17; N, 6.54

¹H NMR Spectra (CDCl₃) (8, ppm): 1.12 (s, 3H, C17-H), 1.26 (t, 3H, C16-H), 1.36 (br, 12H, LysMe γ-CH₂, δ-CH₂), 1.67 (s, 3H, C19-H), 1.79 (br, 6H, LysMe β-CH₂), 1.88 (br, 3H, C18-H), 2.25 (s, 3H, C10C-OAc), 2.41 (s, 3H, C4-OAc), 2.49 (br, 2H, succinyl CH₂), 2.74 (br, 2H, succinyl CH₂), 3.05 (br, 6H, LysMe ε-CH₂), 3.39 (s, 9H, MPEG350 OCH₃), 3.66 (br, 72H, MPEG350 OCH₂CH₂), 4.02 (d, 2H, Gly CH₂), 3.79, (d, 1H, C3-H), 3.95 (br, 12H, MPEG350 OCH₂CH₂), 4.13 (m, 2H, C20-H), 4.27 (d, 1H, C7-H), 4.47 (br, 3H, LysMe α-CH), 4.98 (d, 1H, C5-H), 5.43 (s, 1H, C2′-H), 5.66 (d, 1H, C2-H), 5.90 (m, 1H, C3′-H), 6.17 (m, 1H C13-H), 6.29 (s, 1H, C10-H), 7.39 (m, 3′Ph), 7.46 (br, 3′-NBz), 7.50 (m, 2-OBz), 7.80 (d, 3′-OBZ), 8.12 (d, 2-OBz).

³¹P NMR Spectra (CDCl₃, ppm): δ 22.5

### Example 2

### Preparation of {[tris(methoxy-polyethyleneglycol550)][tris(glycyllysinemethylester)-mono-2′-succinyltaxol]cyclotriphosphazene}, [N₃P₃(MPEG550)₃(GlyLys-Me-2′-succinyltaxol)(GlyLysMe)₂]

The same procedures as described in Example 1 were carried out with methoxy-poly(ethylene glycol) having a molecular weight of 550 (MPEG550) (5.24 g, 9.50 mmol) to obtain the desired product (58% of yield).

Empirical formula: C₁₅₃H₂₅₈N₁₃O₆₅P₃

Molecular weight: 3,318

Elementary analysis (%)

Found: C, 54.22; H, 8.06; N, 5.84

Calculated (%): C, 54.31; H, 7.69; N, 5.47

¹H NMR Spectra (CDCl₃)(δ, ppm): 1.13 (s, 3H, C17-H), 1.20 (t, 3H, C16-H), 1.43 (br, 12H, LysMe γ-CH₂, δ-CH₂), 1.67 (s, 3H, C19-H), 1.79 (br, 6H, LysMe β-CH₂), 1.89 (br, 3H, C18-H), 2.18 (s, 3H, C10-OAc), 2.42 (s, 3H, C4-OAc), 2.49 (br, 2H, succinyl CH₂), 2.74 (br, 2H, succinyl CH₂), 3.10 (br, 6H, LysMe ε-CH₂), 3.38 (s, 9H, MPEG550 OCH₃), 3.65 (br, 132H, MPEG550 OCH₂CH₂), 4.04 (d, 2H, Gly CH₂), 3.18, (d, 1H, C3-H), 4.44 (br, 3H, LysMe α-CH) 4.28 (m, 2H, C20-H), 4.35 (d, 1H, C7-H), 4.99 (d, 1H, C5-H), 5.41 (s, 1H, C2′-H), 5.67 (d, 1H, C2-H), 5.91 (m, 1H, C3′-H), 6.19 (m, 1H C13-H), 6.29 (s, 1H, C10-H), 7.40 (m, 3′Ph), 7.46 (br, 3′-NBz), 7.50 (m, 2-OBz), 7.80 (d, 3′-OBZ), 8.12 (d, 2-OBz).

³¹P NMR Spectra (CDCl₃, ppm): δ22.54

### Example 3

### Preparation of {[tris(methoxy-polyethyleneglycol550)][tris(glycyllysinemethylester)-di-2′-succinyltaxol]cyclotriphosphazene}, [N₃P₃(MPEG550)₃(GlyLys-Me-2′-succinyltaxol)₂(GlyLysMe)]

The same procedures as described in Example 1 were carried out with methoxy-poly(ethylene glycol) having a molecular weight of 550 (MPEG550) (5.24 g, 9.50 mmol) and two fold amount of 2′-succinyltaxol (540 mg, 0.58 mmol) to obtain the desired product (58% of yield).

Empirical formula: C₂₀₄H₃₁₂N₁₄O₈₂P₃

Molecular weight: 4,318

Elementary analysis (%)

Found: C, 54.22; H, 8.06; N, 5.84

Calculated (%): C, 54.74; H, 7.28; N, 4.54

¹H NMR Spectra (CDCl₃)(δ, ppm): 1.13 (s, 3H, C17-H), 1.20 (t, 3H, C16-H), 1.43 (br, 12H, LysMe γ-CH₂, δ-CH₂), 1.67 (s, 3H, C19-H), 1.79 (br, 6H, LysMe β-CH₂), 1.89 (br, 3H, C18-H), 2.18 (s, 3H, C10-OAc), 2.42 (s, 3H, C4-OAc), 2.49 (br, 2H, succinyl CH₂), 2.74 (br, 2H, succinyl CH₂), 3.10 (br, 6H, LysMe ε-CH₂), 3.38 (s, 9H, MPEG550 OCH₃), 3.65 (br, 132H, MPEG550 OCH₂CH₂), 4.04 (d, 2H, Gly CH₂), 3.18, (d, 1H, C3-H), 4.44 (br, 3H, LysMe α-CH) 4.28 (m, 2H, C20-H), 4.35 (d, 1H, C7-H), 4.99 (d, 1H, C5-H), 5.41 (s, 1H, C2′-H), 5.67 (d, 1H, C2-H), 5.91 (m, 1H, C3′-H), 6.19 (m, 1H C13-H), 6.29 (s, 1H, C10-H), 7.40 (m, 3′Ph), 7.46 (br, 3′-NBz), 7.50 (m, 2-OBz), 7.80 (d, 3′-OBZ), 8.12 (d, 2-OBz).

³¹P NMR Spectra (CDCl₃, ppm): δ22.54

### Example 4

### DLS Measurements for Micelle Formation of the Cyclotriphosphazene-taxol Conjugate and the Micelles Size

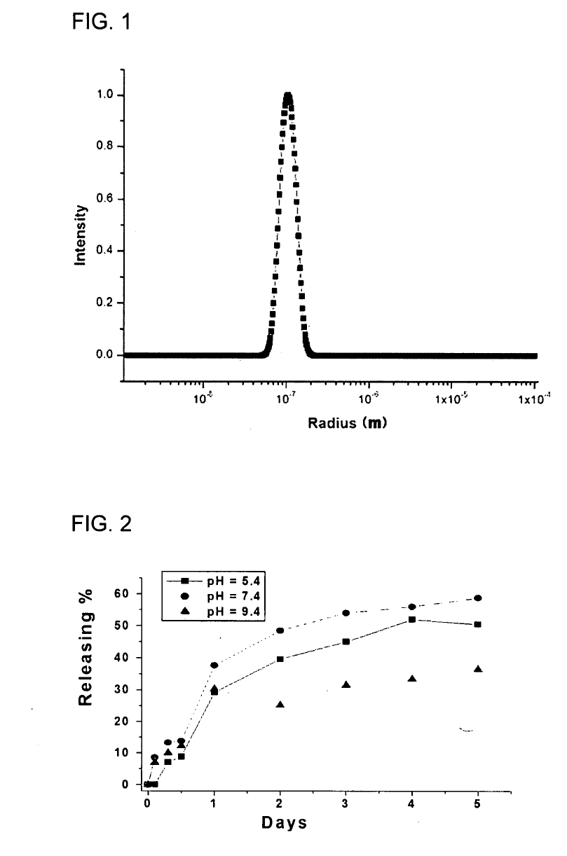

It was determined by a DLS (dynamic light scattering) method whether the cyclotriphosphazene-taxol conjugates prepared according to the present invention are aggregated in aqueous solution through interaction of the hydrophobic oligopeptide groups, so as to form micelles, according to the following procedure: A size distribution of an aqueous solution, in which 0.1 to 0.5% of the cyclotriphosphazene-taxol conjugate prepared in Example 1 was dissolved in distilled water, was measured by a DLS method using Malvern Zetasizer (Nano-ZS). As can be seen from FIG. 1, it was observed that micelles with a radius of 105 nm were formed.

### Example 5

### Degradation Test of Cycloriphosphazene-Taxol Conjugates in a PBS Solution

In order for a cyclotriphosphazene-taxol conjugate to exhibit an in vivo anticancer activity, it is required that taxol molecules be separated via an enzymatic degradation or hydrolysis from the cyclotriphosphazene-taxol conjugate in which taxol molecule is chemically bonded to the cyclotriphosphazene drug delivery material. Therefore, a hydrolysis test in a PBS solution was carried out according to the following procedure.

The cyclotriphosphazene-taxol conjugate from Example 1 (3.4 mg) was dissolved in 0.1 ml of DMSO, and then diluted with 0.9 ml of a PBS solution. While the resulting solution was incubated in a water bath maintained at 37° C., samples were taken from the solution at appropriate intervals. The samples were freeze-dried and then analyzed with HPLC by eluting with a mixed solvent of water and acetonitrile (6:4 by volume) containing 0.1% TFA at a flow rate of 1 ml/min. The same experiment was performed at different pHs (pH 5.4/7.4/9.4) using different buffer solutions, and the results are shown in FIG. 2.

### Example 6

### In vitro Anticancer Activity of Cycloriphosphazene-Taxol Conjugates

In order to evaluate the anticancer activity of the cyclotriphosphazene-taxol conjugates of the present invention, in vitro cytotoxicity of the representative compound of Example 1 was assayed both in saline and in pure water according to a standard testing procedure (Y. J. Jun, et al., J. Inorg. Biochem. (2005) 99:1593-1601), and the test results are shown in Table 1 below.

As can be seen from Table 1, the compound of Example 1 shows low cytotoxicity in pure water probably because of its slow degradation, but much higher cytotoxicity is observed in saline solution similar to the biological fluid due to relatively rapid degradation of the conjugate, releasing the taxol molecules at a reasonably high rate. Since the dipeptide used as a spacer group for the conjugate is easily degraded by peptidases existing in the lysosome of the cell, it is expected that in vivo anticancer activity would be much higher.

TABLE 1
In vitro Cytotoxicity of a Cycloriphosphazene-taxol Conjugate

| | IC50 value (nM) (average ± SD, n = 3 − 4) | | | |
|---|---|---|---|---|
| Compound | MCF-7 | SK-OV3 | A-431 | MDA-MB-231 |
| Taxol (free) | 39.0 ± 4.9 | 65.8 ± 0.5 | 25.1 ± 8.9 | 47.1 ± 12.2 |
| Example 1 | 128.6 ± 37.5 | 137.5 ± 14.3 | 53.8 ± 13.9 | 107.5 ± 10.9 |
| In saline solution | | | | |
| Example 1 | 447.4 ± 138.2 | 503.4 ± 114.3 | 379.9 ± 89.2 | 327.2 ± 54.5 |
| In pure water | | | | |

Note)
MCF-7 and MDA-MB-231 are breast cancer cell lines; SK-OV3 is a ovarian cancer cell line; and A-431 is a cervical cancer cell line.

### Example 7

### Blood Metabolism Study of Cycloriphosphazene-Taxol Conjugates

To a group consisting of 4 male Sprague-Dawley rats (270-300 g) was administered the compound of Example 2 dissolved in a PBS solution at a concentration of 6.67 mg/ml by intravenous infusion with a dosage of 5 mg/kg for 3 minutes. After the drug administration, blood (100 μl) samples were taken from each rat for 48 hours at 0, 0.08, 0.5, 1, 2, 3, 4, 6, 8, 12, 24, 36 and 48 hours post injection, and the samples were analyzed with HPLC, and the results are presented in Table 2 below.

As shown in Table 2, it was discovered that in vivo half life of the compound of Example 2 was twice longer than that of free taxol which was not formulated.

TABLE 2
Metabolism Study of a Cyclotriphosphazene-taxol Conjugate

| | Paclitaxel | | | |
|---|---|---|---|---|
| | (control group) | | Example 2 | |
| Variable | Average | S.D. | Average | S.D. |
| C0 (ug/ml) | 2.70 | 0.65 | 1.53 | 0.15 |
| Lambda z (hr-1) | 0.275 | 0.165 | 0.133 | 0.077 |
| t½_Lambda z (hr) | 3.54 | 2.46 | 6.29 | 2.51 |
| Vz_obs (ml) | 2778.1 | 1735.0 | 4468.9 | 2059.6 |
| Cl_obs (ml/hr) | 556.2 | 63.0 | 513.3 | 145.1 |
| AUClast (ug*hr/ml) | 1.97 | 0.31 | 1.97 | 0.59 |
| AUCINF (measurement) | 2.29 | 0.28 | 2.55 | 0.66 |
| AUClast/AUCINF(%) | 85.9 | 9.4 | 77.1 | 10.5 |

According to the present invention, a micelle-forming cyclotriphosphazene-taxol conjugate anticancer agent capable of being slowly degraded and releasing free taxol in vivo, and a preparation method thereof are provided. The cyclotriphosphazene-taxol conjugate anticancer agent is well dissolved in water, and thus, it can be directly administered by injection in a short time, so as to make the treatment for patients more convenient and less expensive. Micelles are formed in blood upon administering the anticancer agent in vivo, and while circulating in the blood for a long time, taxol molecules are released by enzymatic and hydrolytic degradations from the micelles so that they can be slowly supplied into blood. Therefore, toxicity due to an instant administration of the anticancer agent can fundamentally be reduced, and the pharmacological effects of taxol can continuously be maintained, by which superior anticancer activity can be expected. It can also be expected that the present conjugate drug can be widely used as a new anticancer agent having less adverse effects and drastically improved efficacy.

What is claimed is:

1. A cyclotriphosphazene-taxol conjugate anticancer agent represented by the following Formula 1: 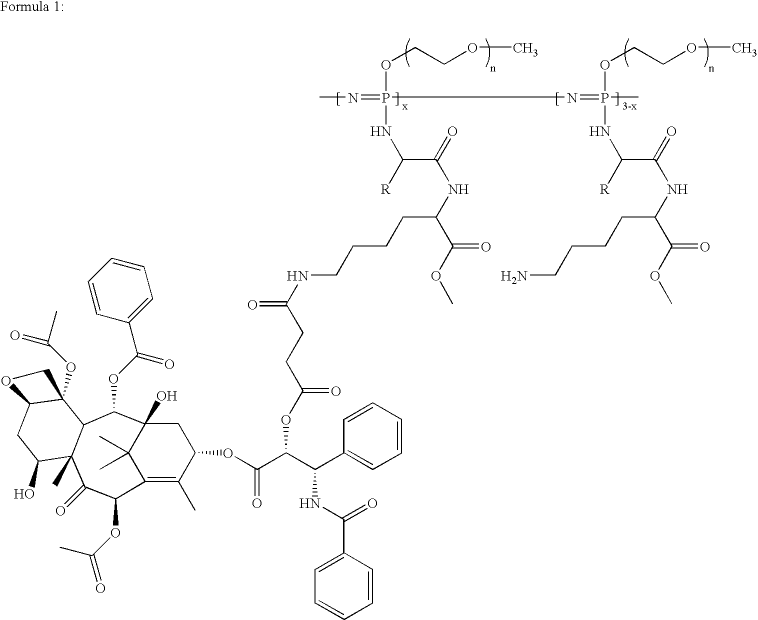
wherein R is selected from the group consisting of H, CH₃, (CH₃)₂CH and (CH₃)₂CHCH₂; each n is the number of the ethylene oxide repeating unit of poly(ethylene glycol) selected from integers of 7-12; and x is 1 or 2.

2. The anticancer agent according to claim 1, forming stable micelles with a mean diameter of 10-150 nm in an aqueous solution.

3. The anticancer agent according to claim 1, wherein molecular weight of methoxy-poly(ethylene glycol) is 350, 550 or 750.

4. A preparation method of a cyclotriphosphazene-taxol conjugate anticancer agent represented by Formula 1, comprising: 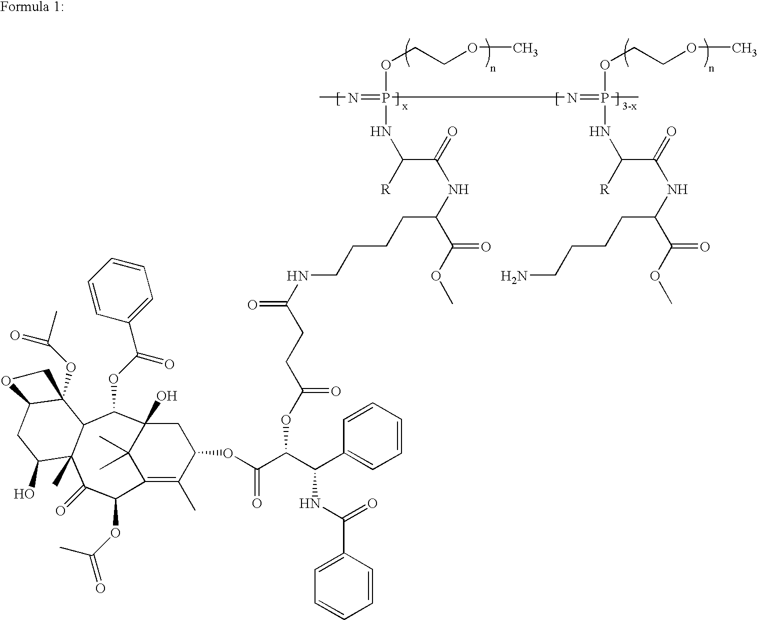
(1) reacting a sodium salt of a poly(ethylene glycol) represented by Formula 3 with hexachlorocyclotriphosphazene represented by Formula 4 to obtain a cyclotriphosphazene intermediate represented by Formula 5: 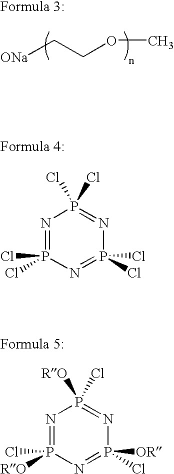
(2) reacting the cyclotriphosphazene intermediate of Formula 5 with a methyl ester of glycyllysine represented by Formula 6 to obtain a compound represented by Formula 7, followed by deprotecting, to obtain a compound represented by Formula 8; and 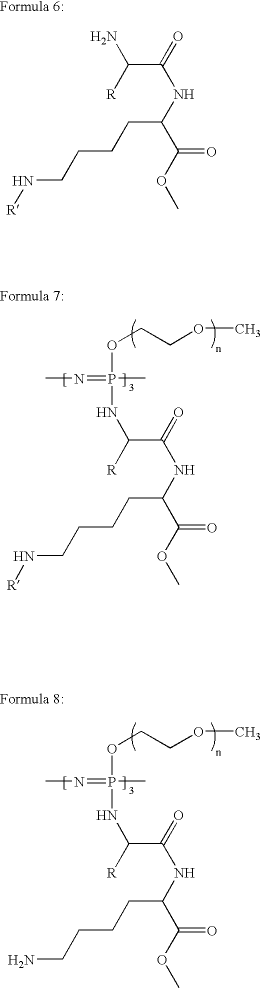
(3) reacting the compound of Formula 8 with succinyl taxol to obtain a cyclrotriphosphazene-taxol conjugate represented by Formula 1,
wherein R is selected from the group consisting of H, CH₃, (CH₃)₂CH and (CH₃)₂CHCH₂; R′ is a benzyloxycarbonyl group or a t-butoxycarbonyl group; R″ is CH₃(OCH₂CH₂)ₙ; each n is the number of ethylene oxide repeating unit of poly(ethylene glycol) selected from integers of 7 to 12, and x is 1 or 2.

5. The method according to claim 4, wherein in step (1), 1 mol of hexachlorocyclotriphosphazene of Formula 4 is reacted with 3-4 equivalents of the sodium salt of Formula 3 in the presence of triethylamine.

6. The method according to claim 4, wherein in step (2), 1.1 to 1.5 equivalents of the compound of Formula 6 for each chlorine atom of three un-substituted chlorine atoms existing in the cyclotriphosphazene intermediate of Formula 5 are reacted with the cyclotriphosphazene intermediate of Formula 5.

7. The method according to claim 4, wherein in step (3), 1 or 2 equivalents of 2′-succinyl taxol are reacted with 1 mole of the compound of Formula 8.

8. The method according to claim 4, wherein in step (3), a combination of 1-ethyl-3-(3-dimethylaminopropyl)carbodiimide and 1-hydroxybenzonitroazole hydrate is used as a reagent for coupling the compound of Formula 8 with the 2′-succinyl taxol.

9. A method for treatment of a cancer, comprising administration of the cyclotriphosphazene-taxol conjugate anticancer agent according to claim 1.
